(19) United States
(12) Patent Application Publication
(10) Pub. No.: US 2026/0364048 A1
(43) Pub. Date: Nov. 19, 2026

(54) COMPOSITION FOR IMPROVING PRECOCIOUS PUBERTY AND USE THEREOF

(71) Applicant: Tadpole Jingyan Biotechnology (Suzhou) Co., Ltd., Suzhou (CN)

(72) Inventors: Hengxiang Cui, Suzhou (CN); Tong Hua, Suzhou (CN)

(21) Appl. No.: 19/379,721

(22) Filed: Nov. 4, 2025

(30) Foreign Application Priority Data
Apr. 29, 2025 (CN) 202510557485.2

(51) Int. Cl. A61K 31/385; A61K 31/121; A61K 31/122; A61K 31/194; A61K 31/23; A61K 31/26; A61K 31/353; A61K 31/4045; A61K 31/593; A61K 31/716; A61K 36/82; A61P 3/04; A61P 5/06; A61P 5/26; A61P 5/30; A61P 15/00

(52) U.S. Cl. CPC A61K 31/385; A61K 31/121; A61K 31/122; A61K 31/194; A61K 31/23; A61K 31/26; A61K 31/353; A61K 31/4045; A61K 31/593; A61K 31/716; A61K 36/82; A61P 3/04; A61P 5/06; A61P 5/26; A61P 5/30; A61P 15/00

(57) ABSTRACT

Disclosed in the present disclosure are a composition for improving precocious puberty and use thereof. The composition includes a first component and a second component; and the first component includes alpha-lipoic acid, and the second component includes sulforaphane and/or alpha-ketoglutaric acid. The composition of the present disclosure can delay symptoms of precocious puberty populations in children, especially obese children related to metabolism, and assist in repairing the mitophagy function of chondrocytes.

### CROSS REFERENCE TO RELATED APPLICATIONS

The present application claims the benefit of Chinese Patent Application No. 202510557485.2 filed on Apr. 29, 2025, the contents of which are incorporated herein by reference in their entirety.

### FIELD OF THE INVENTION

The present disclosure belongs to the technical field of foods, and in particular relates to a composition for improving precocious puberty and use thereof.

### BACKGROUND OF THE INVENTION

Childhood precocious puberty is closely related to obesity, premature estrogen secretion and premature growth plate closure, and the disease usually refers to the appearance of secondary sexual characteristics before 8 years of age in girls and before 9 years of age in boys. Secondary sexual characteristics of girls include breast enlargement, pubic hair and axillary hair growth, menarche, and the like; secondary sexual characteristics of boys include scrotum and testicle enlargement, pubic hair and axillary hair growth, voice change, beard growth, and the like. Precocious puberty can be divided into three categories, i.e., central precocious puberty, peripheral precocious puberty and partial precocious puberty according to whether it depends on Gonadotropin-releasing hormone (GnRH). The peripheral precocious puberty (also known as non-GnRH-dependent or pseudoprecocious puberty) is not controlled by the hypothalamic-pituitary-gonadal axis, and results in early development of second sexual characteristics and increase of sex hormone levels in pediatric patients due to other reasons, but the gonadal axis is immature, and usually, gonads do not develop prematurely. At present, with the improvement of living standards, children may have the habit of a high-fat diet in daily life. The high-fat diet (HFD) can simulate the peripheral precocious puberty (non-GnRH-dependent) by inducing precocious puberty through obesity and increased estrogen secretion.

Mitophagy plays a pivotal role in the development of precocious puberty by integrating energy metabolism, oxidative stress and hormone signaling. Defects in mitophagy may lead to abnormal energy induction; and damaged mitochondrial accumulation triggers ATP deficiency, triggering premature activation of the compensatory HPG axis. Elevated levels of circulating saturated fatty acids associated with the high-fat diets induce neuroinflammation, endoplasmic reticulum stress and hormone resistance in the hypothalamus. Generally, estrogen inhibits PINK1 expression through ER alpha (an estrogen receptor alpha), resulting in a decrease in mitophagy, an increase in estrogen, inhibition of autophagy, and an increase in intracellular reactive oxygen species (ROS), ultimately leading to a feedback loop of accelerated cartilage differentiation.

Childhood precocious puberty, a common disease of the endocrine system in children, has core harms of accelerating bone maturation and limiting adult height, and may cause metabolic, psychological, and long-term health problems. Early diagnosis and comprehensive intervention are the key. By inhibiting sex hormone secretion and delaying the progression of a bone age, the quality of life and health outcomes of pediatric patients can be significantly improved. Based on this, the present disclosure proposes a composition for improving the problem of precocious puberty.

### SUMMARY OF THE INVENTION

In order to solve the above technical problems, an object of the present disclosure is to provide a composition for improving precocious puberty and use thereof. Combining alpha-lipoic acid with sulforaphane and/or alpha-ketoglutaric acid can delay symptoms of precocious puberty populations in children, especially obese children related to metabolism, and assist in repairing the mitophagy function of chondrocytes.

To achieve the above object of the present disclosure, the technical solutions adopted in the present disclosure are as follows:
in a first aspect of the present disclosure, the present disclosure provides a composition for improving precocious puberty, including a first component and a second component, wherein the first component includes alpha-lipoic acid, and the second component includes sulforaphane and/or alpha-ketoglutaric acid.

Preferably, the first component includes 100-300 parts by weight alpha-lipoic acid, and the second component includes 10-400 parts by weight sulforaphane and/or 50-800 parts by weight alpha-ketoglutaric acid.

Preferably, the second component includes sulforaphane and alpha-ketoglutaric acid, i.e., the composition includes three components of alpha-lipoic acid, sulforaphane and alpha-ketoglutaric acid.

Preferably, the composition further includes a third component including β-glucan.

More preferably, the composition includes 500-4000 parts by weight β-glucan.

More preferably, the third component further includes one or more of melatonin, curcumin, quercetin, green tea polyphenols, vitamin and omega-3 fatty acids.

More preferably, the vitamin includes vitamin K2 and/or vitamin D3.

In a second aspect of the present disclosure, the present disclosure provides use of the composition for improving precocious puberty, wherein the above composition is used for ameliorating symptoms of precocious puberty populations and improving chondrocyte mitophagy.

In a third aspect of the present disclosure, the present disclosure provides use of the composition for improving precocious puberty, wherein the composition is prepared into a food, a drug or a health product.

Preferably, the composition is prepared into a liquid beverage, a tablet, a capsule, a solid beverage or a functional food.

Beneficial effects:

Alpha-lipoic acid of the present disclosure is a food raw material or a nutritional fortifier, and such substance is an endogenous metabolite contained in the human body and has an advantage in terms of safety to human cells. Alpha-ketoglutaric acid is also an endogenous metabolite contained in the human body. Sulforaphane is a widely used nutrient. The components of the composition of the present disclosure are highly safe to the human body. After precise adjustment, the composition can give full play to the advantages of each component, better ameliorate the symptoms of precocious puberty, and enhance mitophagy of cells cultured in vitro, and reduce intracellular reactive oxygen species, thereby contributing to the improvement of cell metabolism.

### BRIEF DESCRIPTION OF DRAWINGS

FIG. 1 is a graph showing the change in body weight of mice in each group of Example 1 as a function of time;

FIG. 2 is a comparison graph of body fat rates of 45-day-old mice in each group of Example 1;

FIG. 3 is a schematic diagram of the estrus cycle of mice in each group of Example 1 as a function of time;

FIG. 4 is a comparison graph of the days of vaginal opening of mice in each group of Example 1;

FIG. 5 is a comparison graph of the days of first estrus of mice in each group of Example 1;

FIG. 6 is a comparison graph of the expression of Kiss1 genes in hypothalami and pituitaries of mice in each group of Example 1;

FIG. 7 is a comparison graph of the expression of GnRH1 genes in hypothalami and pituitaries of mice in each group of Example 1;

FIG. 8 is a comparison graph of estradiol levels in the blood of mice in each group of Example 1;

FIG. 9 is a comparison graph of follicle stimulating hormone levels in the blood of mice in each group of Example 1;

FIG. 10 is a comparison graph of luteinizing hormone levels in the blood of mice in each group of Example 1;

FIG. 11 is a graph showing Western blot results of the expression of mitophagy markers PINK1, BNIP3 and BNIP3L in tibial growth plate cartilage tissues of mice in each group of Example 1;

FIG. 12 is a graph showing the between-group comparison of the relative BNIP3 protein expression levels of mice in each group of Example 1;

FIG. 13 is a graph showing the between-group comparison of the relative BNIP3L protein expression levels of mice in each group of Example 1;

FIG. 14 is a graph showing the between-group comparison of the relative PINK1 protein expression level of mice in each group of Example 1.

FIG. 15 is a graph showing the between-group comparison of body weight of mice in each group of Example 2 at 42 days of age.

FIG. 16 is a graph showing the between-group comparison of testosterone levels in serum of mice in each group of Example 2.

FIG. 17 is a graph showing the between-group comparison of leptin levels in serum of mice in each group of Example 2.

FIG. 18 is a graph showing the between-group comparison of the days of prepuce separation of mice in each group of Example 2.

### DETAILED DESCRIPTION OF THE INVENTION

In order to illustrate the examples of the present disclosure or the technical solutions in the prior art more clearly, specific embodiments of the present disclosure will be illustrated below with reference to the accompanying drawings. Obviously, the drawings in the following description are merely some examples of the present disclosure, and for those of ordinary skill in the art, other drawings and other embodiments can also be obtained from these drawings without inventive step.

The present disclosure provides a composition for improving precocious puberty, including a first component and a second component, wherein the first component includes alpha-lipoic acid, and the second component includes sulforaphane and/or alpha-ketoglutaric acid. That is, the composition may be a combination of alpha-lipoic acid and sulforaphane, a combination of alpha-lipoic acid and alpha-ketoglutaric acid, or a combination of sulforaphane, alpha-lipoic acid and alpha-ketoglutaric acid. Preferably, the composition includes alpha-lipoic acid, sulforaphane and alpha-ketoglutaric acid.

Sulforaphane is a natural isothiocyanate compound primarily derived from cruciferous vegetables (such as broccoli seeds) with powerful antioxidant, anti-inflammatory and detoxifying activities.

Alpha-Lipoic Acid (ALA) is a natural dithiol compound contained in the human body. In recent years, research in the fields of antioxidation, metabolic regulation and disease treatment has attracted much attention. Alpha-lipoic acid can promote osteoblast differentiation by activating a Wnt signaling pathway and reduce osteoclast activity by inhibiting RANKL expression, thereby balancing bone formation and bone resorption.

Alpha-ketoglutaric acid (AKG) is an intermediate in a tricarboxylic acid (TCA) cycle, and is involved in various metabolic and cellular pathways. AKG, as an antioxidant, interferes with the balance of nitrogen and ammonia, and affects epigenetics and immune regulation. AKG has potential benefits in muscle growth, wound healing, and promoting faster recovery after surgery.

Sulforaphane, alpha-lipoic acid, alpha-ketoglutaric acid and the like are common food-grade raw materials or food additives or nutritional fortifiers with a wide range of safe concentrations for human cells. The components of alpha-lipoic acid and alpha-ketoglutaric acid themselves are present in the human body, are endogenous metabolites common to humans and various animals, can be directly involved in biochemical processes in human cells, and have an advantage in terms of safety to human cells. However, there is currently no evidence that sulforaphane, alpha-lipoic acid or alpha-ketoglutaric acid has an effect in the treatment of precocity related disorders. In the present disclosure, sulforaphane, alpha-lipoic acid and alpha-ketoglutaric acid can fully exert their respective advantages, more importantly, the present disclosure innovatively uses alpha-lipoic acid as a main component to be combined with sulforaphane and/or alpha-ketoglutaric acid to exert the effect of ameliorating symptoms of precocious puberty, improving puberty initiation and first estrus time, and the expression of GnRH1 and Kiss1 genes in the hypothalamus and pituitary, and synergistically promoting the return of hormones such as estradiol (E2)/testosterone, luteinizing hormone (LH) and follicle stimulating hormone (FSH) in serum of animals to normal steady-state levels.

The present disclosure adopts the first component and the second component as main active components, and the active components mentioned in the present disclosure all refer to the first component and the second component.

Preferably, in the present disclosure, the first component includes 100-300 parts by weight alpha-lipoic acid, and the second component includes 10-400 parts by weight sulforaphane and/or 50-800 parts by weight alpha-ketoglutaric acid. More preferably, alpha-lipoic acid is 100-300 parts by weight, sulforaphane is 20-200 parts by weight and alpha-ketoglutaric acid is 50-500 parts by weight. By precisely adjusting the amounts of the three components used, the effect of improving precocious puberty can be better exerted. The composition of the present disclosure can delay symptoms of precocious puberty populations in children, especially obese children related to metabolism, and assist in repairing the mitophagy function of chondrocytes.

In some examples, the composition for improving precocious puberty further includes a third component, the effect of the third component is similar to that of an auxiliary component, the third component enhances the effect of the first component and the second component in ameliorating the symptoms of precocious puberty, and the third component includes β-glucan.

β-glucan is a polysaccharide formed by connecting glucose molecules via β-1,3-bonds and β-1,6-bonds. β-glucan plays an important role in the immune system. β-glucan can enhance the activity of immune cells, promote proliferation and differentiation of immune cells, and improve the ability of immune cells to recognize and eliminate pathogens. Preferably, the composition includes 500-4000 parts by weight β-glucan.

Using β-glucan in combination with alpha-lipoic acid, sulforaphane and alpha-ketoglutaric acid can better ameliorate the symptoms of precocious puberty.

In some examples, the third component further includes one or more of melatonin, curcumin, quercetin, green tea polyphenols (EGCG), vitamin, and omega-3 fatty acids. The third component is also a conventional biologically active nutrient or nutrient fortifier that can be selectively used depending on a manner of application of the composition.

The above third component may be added alone, or a plurality of third components may be added according to the requirements. In the case of being prepared into a product, the third component is generally added within the amount allowed by the regulation.

For example, melatonin may be added in an amount of 0.5-20 parts by weight (generally added according to the amount allowed by the regulation, the same hereinafter). Curcumin may be added in an amount of 100-1000 parts by weight. Quercetin may be added in an amount of 50-600 parts by weight. The green tea polyphenols may be added in an amount of 100-1200 parts by weight. The omega-3 fatty acids (containing EPA/DHA, etc.) may be added in an amount of 1000-3000 parts by weight. The vitamin is added appropriately as needed.

In the present disclosure, the kind of the vitamin may be diverse, for example, the vitamin may be vitamin K2 and/or vitamin D3.

The proportion of one composition is provided below, and the composition specifically includes by weight:

100-300 mg of alpha-lipoic acid, 10-400 mg of sulforaphane, 50-800 mg of alpha-ketoglutaric acid, 500-4000 mg of β-glucan, 0.5-20 mg of melatonin, 100-1000 mg of curcumin, 50-600 mg of quercetin, 100-1200 mg of green tea polyphenols (EGCG), 50-400 μg of vitamin K2, 1000-5000 IU vitamin D3, and 1000-3000 mg of omega-3 fatty acids (EPA/DHA). It will be readily appreciated that the third component is added as needed, one or more of the third components may be added, and the usage amounts of the components of the composition may be increased or decreased proportionally.

The application of the composition of the present disclosure is diverse, and the composition can be mixed with other food-grade supplements or nutritional supplements to adjust the taste, and can be prepared into liquid beverages (such as oral liquids), tablets, capsules, solid beverages, and can also be prepared into various functional foods. Several of these product forms are provided below.

Solid components in the first component, the second component and the third component are uniformly mixed and treated by a nano-micronization technology to prepare powder particles having a particle size of 80 μm or less; liquid components in the first component, the second component and the third component are embedded by spray drying to form composite microcapsules; the powder particles and the composite microcapsules are uniformly mixed to form a product, or the powder particles and the composite microcapsules are homogeneously mixed with auxiliary materials such as probiotics and fructo-oligosaccharides under the protection of inert gas, and then the obtained mixture is tableted or encapsulated; and the powder particles and the composite microcapsules can also be prepared into a liquid beverage.

It is readily understood that products formed based on the composition of the present disclosure, without inventive step, should fall within the scope of protection of the present disclosure.

The effect of ameliorating the symptoms of precocious puberty by the composition of the present disclosure is illustrated below by specific experiments.

### Example 1

The effects of the composition of the present disclosure were evaluated by using an HFD-induced mouse precocious puberty model in the experiment.

1. Animals, animal feeding and grouping.

The experiment was performed by using female C57BL/6J mice, and the experiment was divided into a total of 6 groups, with 6 mice in each group. The experimental groups were as follows:
(1) Normal diet (10% energy from fat), recorded as a control group;
(2) High-fat group, high-fat diet (60% energy from fat), recorded as an HFD group;
(3) Nutritional intervention group 1: high-fat diet (60% energy from fat), intervention with Alpha-Lipoic Acid (ALA), recorded as an HFD+ALA group;
(4) Nutritional intervention group 2: high-fat diet (60% energy from fat), combined intervention with alpha-lipoic acid and sulforaphane, recorded as an HFD+ALA+SFN group;
(5) Nutritional intervention group 3: high-fat diet (60% energy from fat), combined intervention with alpha-lipoic acid and alpha-ketoglutaric acid, recorded as an HFD+ALA+AKG group;
(6) Nutritional intervention group 4: high-fat diet (60% energy from fat), combined intervention with alpha-lipoic acid, sulforaphane and alpha-ketoglutaric acid, recorded as an HFD+ALA+SFN+AKG group;

During feeding, from birth to weaning (0-21 days old), female mice were fed with a standard feed, and young mice were naturally suckled; and after weaning (22-45 days old), each group was given the corresponding feed and intervention separately for 24 days.

2. Feed and intervention scheme.

(1) Feed formulation

Control group: standard rodent feed (10% energy from D12450J fat), regularly drinking sterilized water.

HFD group: high-fat feed (60% energy from D12450J fat), regularly drinking sterilized water.

HFD+ALA group: high-fat feed (the same as above), drinking water containing alpha-lipoic acid, with 3.5 mg of alpha-lipoic acid dissolved per 35 ml of drinking water;

HFD+ALA+SFN group: high-fat feed (the same as above), drinking water containing alpha-lipoic acid and sulforaphane, with 3.5 mg of alpha-lipoic acid and 5.25 mg of sulforaphane dissolved per 35 ml of drinking water;

HFD+ALA+AKG group: high-fat feed (the same as above), drinking water containing alpha-lipoic acid and alpha-ketoglutaric acid, with 3.5 mg of alpha-lipoic acid and 7 mg of alpha-ketoglutaric acid dissolved per 35 ml of drinking water;

HFD+ALA+SFN+AKG group: high-fat feed (the same as above), drinking water containing alpha-lipoic acid, sulforaphane and alpha-ketoglutaric acid, with 3.5 mg of alpha-lipoic acid, 7 mg of alpha-ketoglutaric acid and 5.25 mg of sulforaphane dissolved per 35 ml of drinking water;

(2) Nutritional intervention

In the experiment, alpha-lipoic acid, sulforaphane and alpha-ketoglutaric acid were dissolved separately in sterilized water according to the composition scheme of each intervention group specified in “Feed Formulation”, then filtered by using a 0.22 micron ultrafiltration membrane, and supplied to the mice as drinking water, each mouse being supplied with 35 ml of drinking water per day for intervention. For the control group and the HFD group, after the sterilized water was filtered with the 0.22 micron ultrafiltration membrane, the filtered sterilized water was supplied to the mice as drinking water; and 35 ml of drinking water was supplied to each mouse per day. Drinking water (drinking water for intervention) used in all the groups was reconfigured daily.

3. Indicators of experimental analysis

(1) Core indicators for analyzing precocious puberty

Vaginal opening time: the vaginal opening time is a marker of sexual maturity. Observations were performed daily from 21 days old, and the days of vaginal opening were recorded.

Sex hormone detection: orbital blood was collected before sacrifice at 45 days old. Estradiol (E2), luteinizing hormone (LH) and follicle stimulating hormone (FSH) in serum were detected by ELISA.

(2) Metabolism and growth related indicators

Body weight gain curve: the mice were weighed weekly and a growth curve was plotted.

(3) Molecular mechanism evaluation

Gene expression in the hypothalamic-pituitary axis: hypothalamus and pituitary tissues of the mice were taken, and the expression of GnRH and Kiss1 genes in each group was detected by qPCR.

(4) Mitophagy markers: a ratio of PINK1 to BNIP3 to BNIP3L in growth plate cartilage tissue was detected by Western blot.

4. Experimental procedures

Stage 1: Establishment and intervention of HFD precocious puberty model (22-45 days old)

(1) Grouping and feed distribution: after weaning at 21 days old, the mice were randomly grouped according to the body weight to avoid differences in initial body weight between groups;
the mice in the control group were fed with the standard rodent feed (10% energy from D12450J fat), and regularly drank sterilized water;
the mice in the HFD group were fed with the high-fat feed (60% energy from D12450J fat), and regularly drank sterilized water;
the mice in the HFD+ALA group were fed with the high-fat feed (60% energy from D12450J fat), and drank water containing alpha-lipoic acid;
the mice in the HFD+ALA+SFN group were fed with the high-fat feed (60% energy from D12450J fat), and drank water containing alpha-lipoic acid and sulforaphane;
the mice in the HFD+ALA+AKG group were fed with the high-fat feed (60% energy from D12450J fat), and drank water containing alpha-lipoic acid and alpha-ketoglutaric acid;
the mice in the HFD+ALA+SFN+AKG group were fed with the high-fat feed (60% energy from D12450J fat), and drank water containing alpha-lipoic acid, sulforaphane and alpha-ketoglutaric acid;

(2) Daily operation: food intake and body weight were recorded (avoiding stress and weighing at a fixed time); and vaginal opening was observed.

Stage 2: Sample collection and treatment (45 days old)

(1) Anesthesia and sacrifice: pentobarbital sodium (50 mg/kg) was intraperitoneally injected for anesthesia, and the mice were sacrificed by cervical dislocation after blood collection.

(2) Tissue sampling: hypothalami and pituitaries, and tibial growth plate cartilages were rapidly isolated; and then the tissues were quick-frozen in liquid nitrogen (stored at −80 °C).

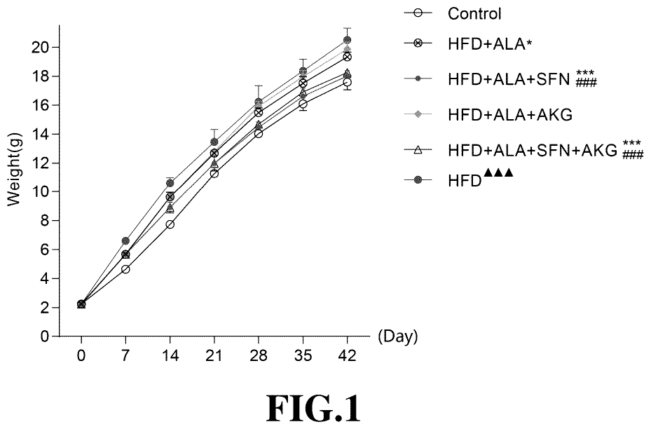

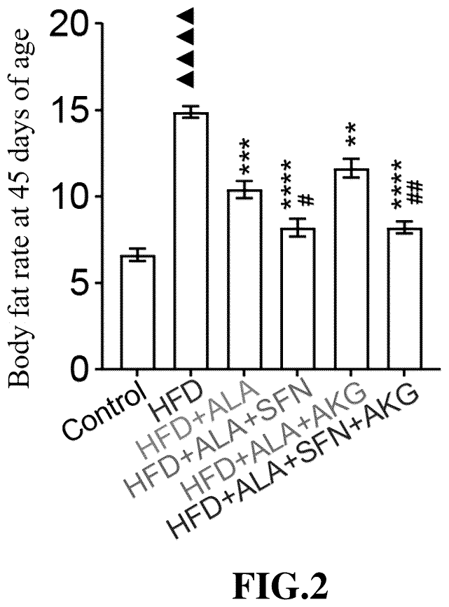

In the female mouse model, the change in body weight of mice as a function of time is shown in FIG. 1. In FIG. 1, a two-way ANOVA test was used for the comparison between groups. As can be seen from FIG. 1, the HFD+ALA+SFN+AKG group, the HFD+ALA+SFN group and the control group had similar body weight changes. The body fat rate of 45-day-old mice is shown in FIG. 2. Data of FIG. 2 was tested by using a Student's t-test. As can be seen from FIG. 2, the HFD+ALA+SFN+AKG group and the HFD+ALA+SFN group had a body fat rate similar to that of the control group. It is indicated that the active components can reduce the effect of the high-fat diet on the body weight.

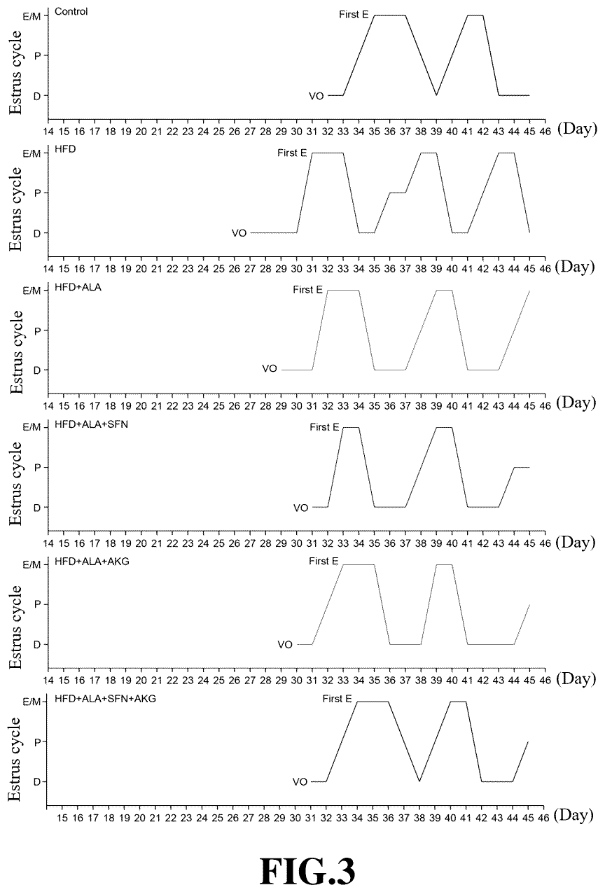

A schematic diagram of the estrus cycle of mice as a function of time is shown in FIG. 3, wherein the ordinate D represents diestrus, the ordinate P represents proestrus, the ordinate E/M represents estrus/metestrus, VO represents the day of first vaginal opening, and First E represents the day of first estrus.

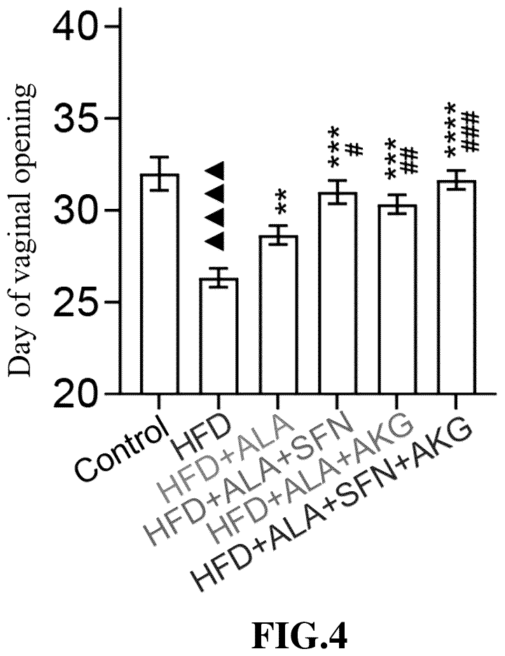

A comparison graph of the days of vaginal opening of mice is shown in FIG. 4. The data in FIG. 4 was tested by using a Log-rank test.

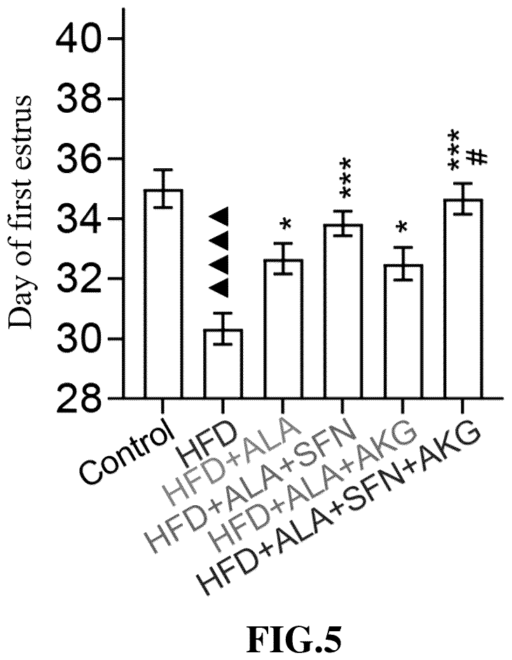

A comparison graph of the days of first estrus of mice is shown in FIG. 5. The data in FIG. 5 was tested by using a Log-rank test.

In FIGS. 4 and 5, ▴ indicates the comparison between the Control group and the HFD group; * indicates the comparisons between the HFD group and the HFD+ALA group, between the HFD group and the HFD+ALA+SFN group, and between the HFD group and the HFD+ALA+AKG group; # indicates the comparisons between the HFD+ALA group and the HFD+ALA+SFN group, and the HFD+ALA group and the HFD+ALA+AKG group; ▴▴▴▴ or **** or #### refers to P<0.0001, ▴▴▴ or *** or ### refers to P<0.001, ▴▴ or ** or ## refers to P<0.01, and ▴ or * or # refers to P<0.05.

As can be seen from the results of FIG. 1, the use of alpha-lipoic acid alone could significantly inhibit HFD-induced weight gain; sulforaphane or sulforaphane in combination with alpha-ketoglutaric acid could significantly synergistically enhance this inhibitory effect of alpha-lipoic acid (FIG. 1). At 45 days of age, it was observed that the use of alpha-lipoic acid alone could significantly inhibit the HFD-induced body fat rate, and sulforaphane or sulforaphane in combination with alpha-ketoglutaric acid could significantly synergistically enhance this inhibitory effect of alpha-lipoic acid (FIG. 2). As can be seen in connection with FIGS. 3-5, the days of vaginal opening of the mice under HFD induction were significantly advanced compared with the Control group; the use of alpha-lipoic acid alone could significantly delay the HFD-induced advance effect of the days of vaginal opening of the mice; and three ways of sulforaphane, alpha-ketoglutaric acid or sulforaphane in combination with alpha-ketoglutaric acid all could significantly synergistically enhance this delayed effect of alpha-lipoic acid (FIGS. 3 and 4). The days of first estrus of the mice under HFD induction were significantly advanced compared with the Control group; the use of alpha-lipoic acid alone could significantly delay the HFD-induced advance effect of the days of first estrus of the mice; and sulforaphane in combination with alpha-ketoglutaric acid could significantly synergistically enhance this delayed effect of alpha-lipoic acid.

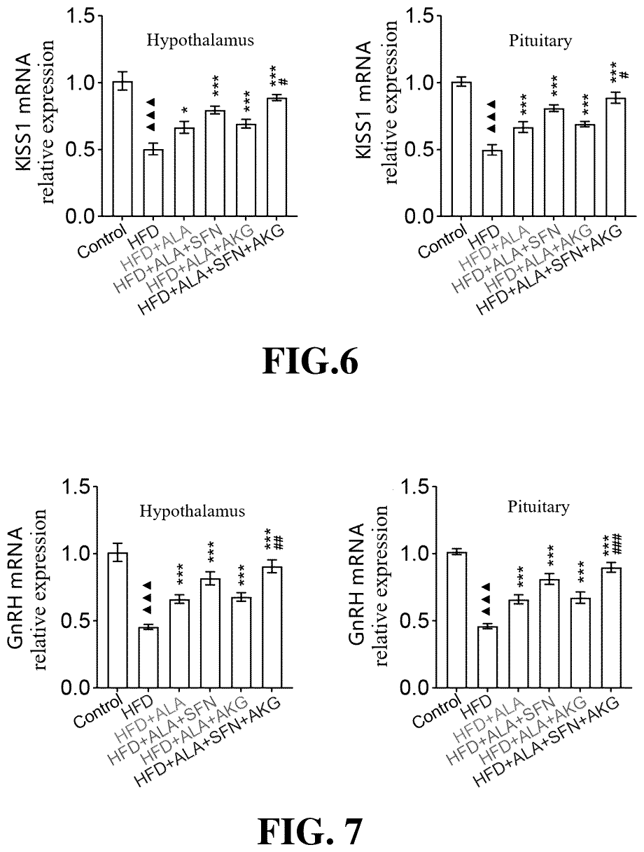

FIG. 6 and FIG. 7 respectively show the expression of GnRH1 and Kiss1 genes in the hypothalami and pituitaries of the mice in each group. FIGS. 6 and 7 show that some or all of the active components exhibit synergistic regulation of the expression of GnRH1 (NM_001416514.1) and Kiss1 (NM_001412624.1) genes in the hypothalami and pituitaries of the mice. A detection method was qRT-PCR, and a β-actin (NM_007393.5) gene was used as an internal reference gene. Data in the HFD+ALA+SFN+AKG group was the best and was closest to that of the Control group.

The Student's t-test was used for the comparisons between groups in FIGS. 6 and 7. ▴ indicates the comparison between the Control group and the HFD group; * indicates the comparisons between the HFD group and the HFD+ALA group, between the HFD group and the HFD+ALA+SFN group, and between the HFD group and the HFD+ALA+AKG group; # indicates the comparisons between the HFD+ALA group and the HFD+ALA+SFN group, and the HFD+ALA group and the HFD+ALA+AKG group; ▴▴▴▴ or **** or #### refers to P<0.0001, ▴▴▴ or *** or ### refers to P<0.001, ▴▴ or ** or ## refers to P<0.01, and ▴ or * or # refers to P<0.05.

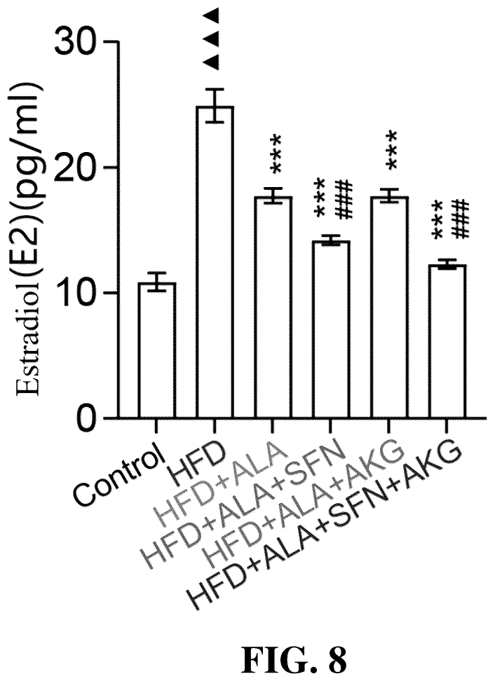

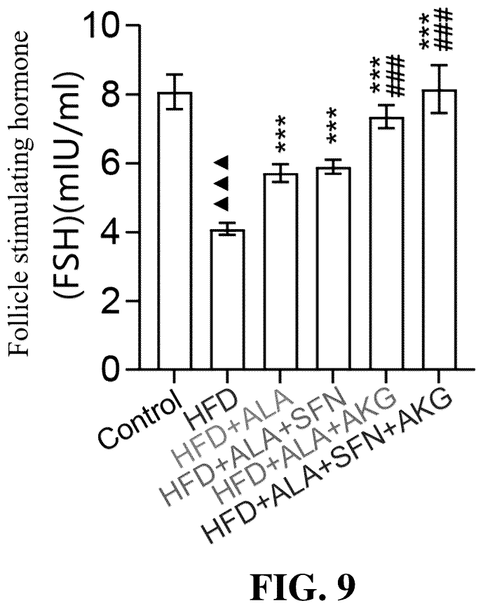

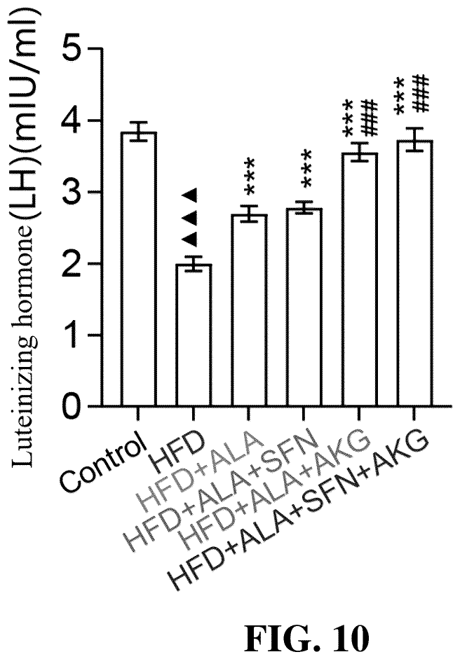

FIGS. 8-10 are comparison graphs of the expression of estradiol (E2), follicle stimulating hormone (FSH), and luteinizing hormone (LH) in the blood of the mice in each group, respectively, showing that some or all of the active components exhibit synergistic regulation of the expression of estradiol, luteinizing hormone, and follicle stimulating hormone in the blood of the mice. Data in the HFD+ALA+SFN+AKG group was the best and was closest to that of the Control group.

The Student's t-test was used for the comparisons between groups in FIGS. 8-10. ▴ indicates the comparison between the Control group and the HFD group; * indicates the comparison between the HFD group and the HFD+ALA group, between the HFD group and the HFD+ALA+SFN group, and between the HFD group and the HFD+ALA+AKG group; # indicates the comparison between the HFD+ALA group and the HFD+ALA+SFN group, and the HFD+ALA group and the HFD+ALA+AKG group; ▴▴▴▴ or **** or #### refers to P<0.0001, ▴▴▴ or *** or ### refers to P<0.001, ▴▴ or ** or ## refers to P<0.01, and ▴ or * or # refers to P<0.05.

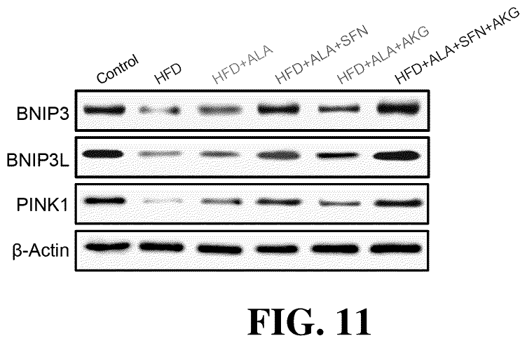

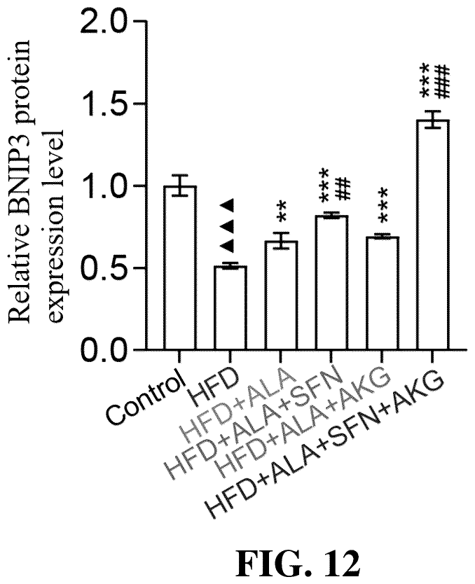

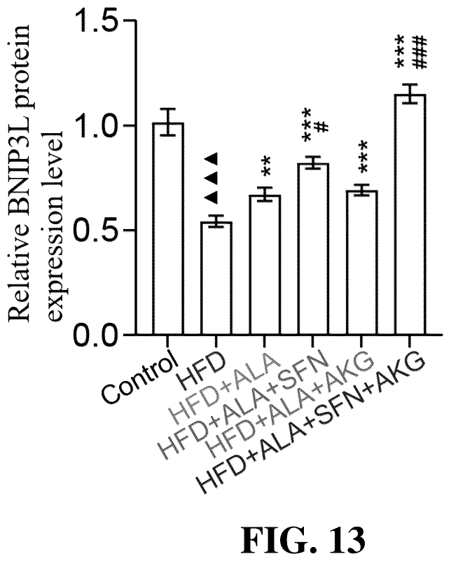

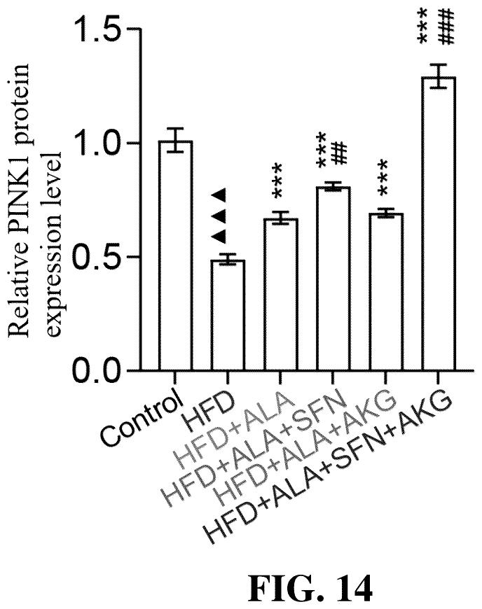

FIG. 11 is a Western blot comparison graph of the expression of mitophagy markers PINK1, BNIP3, and BNIP3L in tibial growth plate cartilage tissues of the mice in each group. FIGS. 12-14 are graphs showing the between-groups comparisons of the relative BNIP3 protein expression level, the relative BNIP3L protein expression level, and the relative PINK1 protein expression level in the mice of each group, respectively. FIGS. 11-14 show that some or all of the active components exhibit synergistic regulation of the expression of estradiol, luteinizing hormone, and follicle stimulating hormone in the blood of the mice. Data in the HFD+ALA+SFN+AKG group was the best.

The Western blot in FIG. 11 shows the expression of the mitophagy markers PINK1, BNIP3 and BNIP3L in the tibial growth plate cartilage tissues of the mice in each treatment group. FIGS. 12-14 show the statistical results of all Western blot experiments performed on the tibial growth plate cartilage tissues of the mice, and the Student's t-test was used for the comparisons between groups. ▴ indicates the comparison between the Control group and the HFD group; * indicates the comparisons between the HFD group and the HFD+ALA group, between the HFD group and the HFD+ALA+SFN group, and between the HFD group and the HFD+ALA+AKG group; #indicates the comparison between the HFD+ALA group and the HFD+ALA+SFN group, and the HFD+ALA group and the HFD+ALA+AKG group; ▴▴▴▴ or **** or #### refers to P<0.0001, ▴▴▴ or *** or ### refers to P<0.001, ▴▴ or ** or ## refers to P<0.01, and ▴ or * or # refers to P<0.05.

From the data shown in FIGS. 1-14, it can be seen that sulforaphane, alpha-lipoic acid and alpha-ketoglutaric acid in the active components of the present disclosure have synergistic effects, and combined use of the three can effectively ameliorate the symptoms of precocious puberty caused by a high-fat diet.

### Example 2

The effects of the composition of the present disclosure were evaluated by using an HFD-induced mouse precocious puberty model in the experiment.

1. Animals, animal feeding and grouping.

The experiment was performed by using male C57BL/6J mice, and the experiment was divided into a total of 6 groups, with 6 mice in each group. The experimental groups were as follows:
(1) Normal diet (10% energy from fat), recorded as a control group;
(2) High-fat group, high-fat diet (60% energy from fat), recorded as an HFD group;
(3) Nutritional intervention group 4: high-fat diet (60% energy from fat), combined intervention with alpha-lipoic acid, sulforaphane and alpha-ketoglutaric acid, recorded as an HFD+ALA+SFN+AKG group;
(4) Nutritional intervention group 5: high-fat diet (60% energy from fat); intervention with the following combination: alpha-lipoic acid, sulforaphane, and alpha-ketoglutaric acid in combination with beta-glucan (β-Glucan), recorded as an HFD+ALA+SFN+AKG+plus1 group;
(5) Nutritional intervention group 6: high-fat diet (60% energy from fat); intervention with the following combination: alpha-lipoic acid, sulforaphane, and alpha-ketoglutaric acid in combination with beta-glucan, curcumin, vitamin K2, vitamin D3 and omega-3 fatty acids, recorded as an HFD+ALA+SFN+AKG+plus2 group;
(6) Nutritional intervention group 7: high-fat diet (60% energy from fat); intervention with the following combination: alpha-lipoic acid, sulforaphane, and alpha-ketoglutaric acid in combination with beta-glucan, curcumin, quercetin, vitamin K2, vitamin D3 and omega-3 fatty acids, recorded as an HFD+ALA+SFN+AKG+plus3 group;
(7) Nutritional intervention group 8: high-fat diet (60% energy from fat); intervention with the following combination: alpha-lipoic acid, sulforaphane, and alpha-ketoglutaric acid in combination with beta-glucan, curcumin, quercetin, melatonin, vitamin K2, vitamin D3 and omega-3 fatty acids, recorded as an HFD+ALA+SFN+AKG+plus4 group;
(8) Nutritional intervention group 9: high-fat diet (60% energy from fat); intervention with the following combination: alpha-lipoic acid, sulforaphane, alpha-ketoglutaric acid, beta-glucan, curcumin, quercetin, melatonin, green tea polyphenols (EGCG), vitamin K2, vitamin D3 and omega-3 fatty acids, recorded as an HFD+ALA+SFN+AKG+plus5 group;

During feeding, from birth to weaning (0-21 days old), female mice were fed with a standard feed, and young mice were naturally suckled; and after weaning (22-45 days old), each group was given the corresponding feed and intervention separately for 24 days.

2. Feed and intervention scheme.

(1) Feed formulation

Control group: standard rodent feed (10% energy from D12450J fat), regularly drinking sterilized water.

HFD group: high-fat feed (60% energy from D12450J fat), regularly drinking sterilized water.

HFD+ALA+SFN+AKG group: high-fat feed (the same as above), drinking water containing alpha-lipoic acid, sulforaphane and alpha-ketoglutaric acid, with 1.8 mg of alpha-lipoic acid, 3.5 mg of alpha-ketoglutaric acid and 2.62 mg of sulforaphane dissolved per 35 ml of drinking water;

HFD+ALA+SFN+AKG+plus1 group: high-fat feed (the same as above), drinking water containing alpha-lipoic acid, sulforaphane and alpha-ketoglutaric acid, with 1.8 mg of alpha-lipoic acid, 3.5 mg of alpha-ketoglutaric acid and 2.62 mg of sulforaphane, and 200 mg of β-glucan (β-1,3-glucan, having a molecular weight of about 500,000 Daltons) dissolved per 35 ml of drinking water;

HFD+ALA+SFN+AKG+plus2 group: high-fat feed (the same as above), drinking water containing alpha-lipoic acid, sulforaphane and alpha-ketoglutaric acid, with 1.8 mg of alpha-lipoic acid, 3.5 mg of alpha-ketoglutaric acid and 2.62 mg of sulforaphane, 200 mg ofβ-glucan (the same as above), 5 mg of curcumin, 0.1 mg of vitamin K2, 0.5 mg of vitamin D3 and 20 mg of omega-3 fatty acids dissolved per 35 ml of drinking water;

HFD+ALA+SFN+AKG+plus3 group: high-fat feed (the same as above), drinking water containing alpha-lipoic acid, sulforaphane and alpha-ketoglutaric acid, with1.8 mg of alpha-lipoic acid, 3.5 mg of alpha-ketoglutaric acid and 2.62 mg of sulforaphane, 200 mg ofβ-glucan (the same as above), 5 mg of curcumin, 5 mg of quercetin, 0.1 mg of vitamin K2, 0.5 mg of vitamin D3 and 20 mg of omega-3 fatty acids dissolved per 35 ml of drinking water;

HFD+ALA+SFN+AKG+plus4 group: high-fat feed (the same as above), drinking water containing alpha-lipoic acid, sulforaphane and alpha-ketoglutaric acid, with 1.8 mg of alpha-lipoic acid, 3.5 mg of alpha-ketoglutaric acid and 2.62 mg of sulforaphane, 200 mg ofβ-glucan (the same as above), 5 mg of curcumin, 5 mg of quercetin, 0.2 mg of melatonin, 0.1 mg of vitamin K2, 0.5 mg of vitamin D3 and 20 mg of omega-3 fatty acids dissolved per 35 ml of drinking water;

HFD+ALA+SFN+AKG+plus5 group: high-fat feed (the same as above), drinking water containing alpha-lipoic acid, sulforaphane and alpha-ketoglutaric acid, with 1.8 mg of alpha-lipoic acid, 3.5 mg of alpha-ketoglutaric acid and 2.62 mg of sulforaphane, 200 mg ofβ-glucan (the same as above), 5 mg of curcumin, 5 mg of quercetin, 0.2 mg of melatonin, 5 mg of green tea polyphenols (EGCG), 0.1 mg of vitamin K2, 0.5 mg of vitamin D3 and 20 mg of omega-3 fatty acids dissolved per 35 ml of drinking water;

In this example, in order to evaluate whether the third components (β-glucan, curcumin, quercetin, vitamin K2, vitamin D3 and omega-3 fatty acids) have a synergistic effect, the selected core components, namely alpha-lipoic acid, sulforaphane and alpha-ketoglutaric acid, were halved in usage amount compared with Example 1.

(2) Nutritional intervention

In the experiment, intervention components in each group were dissolved separately in sterilized water according to the composition scheme of each intervention group specified in “Feed Formulation” in this section, then filtered by using a 0.22 micron ultrafiltration membrane, and supplied to the mice as drinking water, each mouse being supplied with 35 ml of drinking water per day for intervention. For the Control group and the HFD group, after the sterilized water was filtered with the 0.22 micron ultrafiltration membrane, the filtered sterilized water was supplied to the mice as drinking water; and 35 ml of drinking water was supplied to each mouse per day. Drinking water (intervention drinking water) used in all the groups was reconfigured daily.

3. Indicators of experimental analysis

(1) Core indicators for analyzing precocious puberty

Prepuce separation (PPS) time: whether a glans penis was isolated from a prepuce was examined daily from 21 days old (positive for first exposure of the glans penis), and the day of prepuce separation (the day of PPS) of each mouse was recorded.

Sex hormone detection: starting from on the day of prepuce separation, microvenous blood was taken on the same day. Testosterone and Leptin in serum were detected by ELISA.

4. Experimental procedures

Stage 1: Establishment and intervention of HFD precocious puberty model (22-45 days old)

(1) Grouping and feed distribution: after weaning at 21 days old, the mice were randomly grouped according to the body weight to avoid differences in initial body weight between groups;
the mice in the control group were fed with the standard rodent feed (10% energy from D12450J fat), and regularly drank sterilized water;
the mice in the HFD group were fed with the high-fat feed (60% energy from D12450J fat), and regularly drank sterilized water;
the mice in the HFD+ALA+SFN+AKG group were fed with the high-fat feed (60% energy from D12450J fat), and drank water containing alpha-lipoic acid, sulforaphane and alpha-ketoglutaric acid;
the mice in the HFD+ALA+SFN+AKG+Plus1 group was fed with the high-fat feed (60% energy from D12450J fat), and drank water containing alpha-lipoic acid, sulforaphane and alpha-alpha-ketoglutaric acid, and β-glucan;
the mice in theHFD+ALA+SFN+AKG+plus2 group was fed with the high-fat feed (the same as above), and drank water containing alpha-lipoic acid, sulforaphane and alpha-ketoglutaric acid, β-glucan, curcumin, vitamin K2, vitamin D3 and omega-3 fatty acids;
the mice in theHFD+ALA+SFN+AKG+plus3 group was fed with the high-fat feed (the same as above), and drank water containing alpha-lipoic acid, sulforaphane and alpha-ketoglutaric acid, β-glucan (the same as above), curcumin, quercetin, vitamin K2, vitamin D3 and omega-3 fatty acids;
the mice in theHFD+ALA+SFN+AKG+plus4 group was fed with the high-fat feed (the same as above), and drank water containing alpha-lipoic acid, sulforaphane and alpha-ketoglutaric acid, β-glucan (the same as above), curcumin, quercetin, melatonin, vitamin K2, vitamin D3 and omega-3 fatty acids;
the mice in theHFD+ALA+SFN+AKG+plus5 group was fed with the high-fat feed (the same as above), and drank water containing alpha-lipoic acid, sulforaphane and alpha-ketoglutaric acid, β-glucan (the same as above), curcumin, quercetin, melatonin, green tea polyphenols (EGCG), vitamin K2, vitamin D3 and omega-3 fatty acids;

(2) Daily operation: food intake and body weight were recorded (avoiding stress and weighing at a fixed time); prepuce separation was observed; and microvenous blood was taken on the day of prepuce separation for analysis of testosterone and leptin in serum.

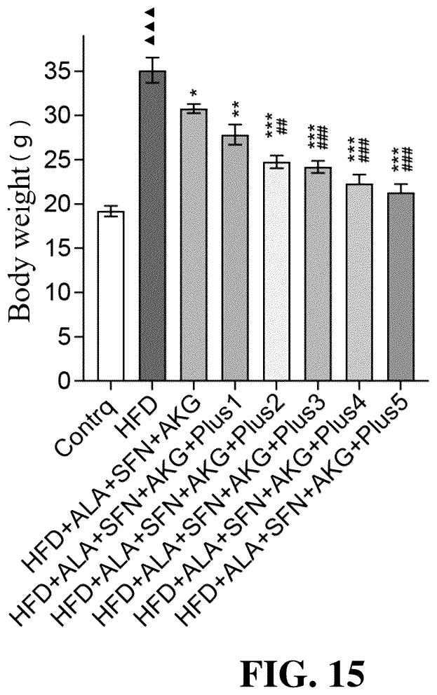

As shown in FIG. 15, in the high-fat (HFD)-induced male mouse precocious puberty model, a combination of alpha-lipoic acid, sulforaphane and alpha-ketoglutaric acid can significantly reduce the body weight of animals; β-glucan in combination with curcumin, vitamin K2, vitamin D3 and omega-3 fatty acids (Plus2 regimen), β-glucan in combination with curcumin, quercetin, vitamin K2, vitamin D3 and omega-3 fatty acids (Plus3 regimen), β-glucan in combination with curcumin, quercetin, melatonin, vitamin K2, vitamin D3 and omega-3 fatty acids (Plus4 regimen), and β-glucan in combination with curcumin, quercetin, melatonin, green tea polyphenols, vitamin K2, vitamin D3 and omega-3 fatty acids (Plus5 regimen) can all enhance this effect.

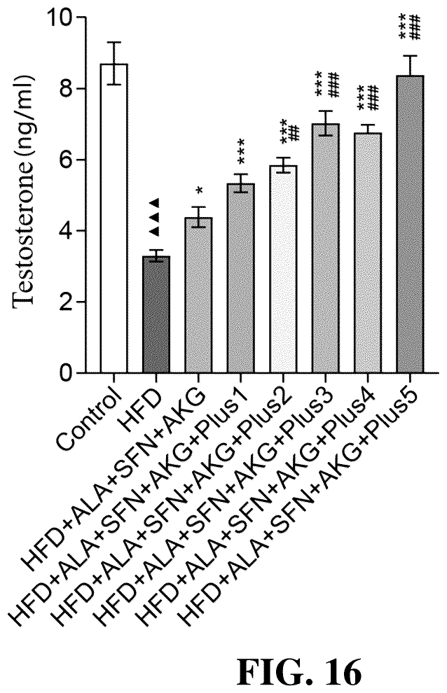

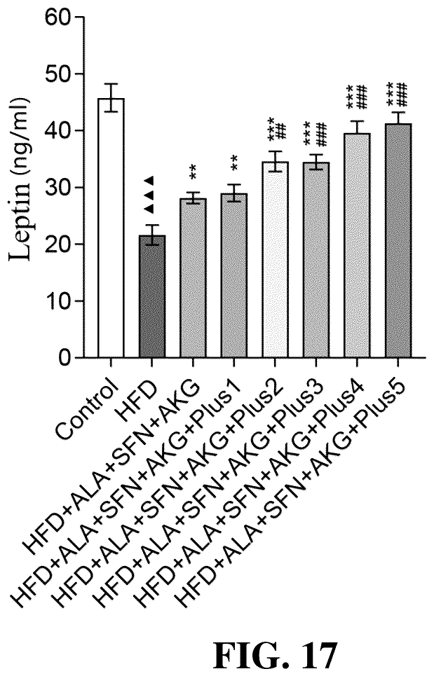

As shown in FIG. 16, testosterone levels in mouse serum were significantly decreased under HFD induction; combined intervention with alpha-lipoic acid, sulforaphane and alpha-ketoglutaric acid can significantly restore the testosterone content in the mouse serum; and β-glucan in combination with curcumin, vitamin K2, vitamin D3 and omega-3 fatty acids (Plus2 regimen), β-glucan in combination with curcumin, quercetin, vitamin K2, vitamin D3 and omega-3 fatty acids (Plus3 regimen), β-glucan in combination with curcumin, quercetin, melatonin, vitamin K2, vitamin D3 and omega-3 fatty acids (Plus4 regimen) and β-glucan in combination with curcumin, quercetin, melatonin, green tea polyphenols, vitamin K2, vitamin D3 and omega-3 fatty acids (Plus5 regimen) can all synergistically enhance this effect. At the same time, as shown in FIG. 17, leptin levels in the mouse serum were significantly decreased under HFD induction; combined intervention with alpha-lipoic acid, sulforaphane and alpha-ketoglutaric acid can significantly restore the leptin content in the mouse serum; and β-glucan in combination with curcumin, vitamin K2, vitamin D3 and omega-3 fatty acids (Plus2 regimen), β-glucan in combination with curcumin, quercetin, vitamin K2, vitamin D3 and omega-3 fatty acids (Plus3 regimen), β-glucan in combination with curcumin, quercetin, melatonin, vitamin K2, vitamin D3 and omega-3 fatty acids (Plus4 regimen) and β-glucan in combination with curcumin, quercetin, melatonin, green tea polyphenols, vitamin K2, vitamin D3 and omega-3 fatty acids (Plus5 regimen) can all synergistically enhance this effect. The Student's t-test was used for the comparisons between groups in FIGS. 15-17;

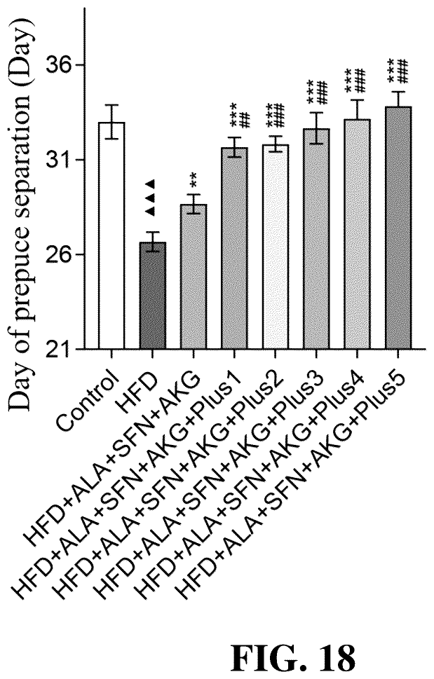

FIG. 18 showed that the days of prepuce separation (the days of PPS) of the male mice were significantly advanced under HFD induction; combined intervention with alpha-lipoic acid, sulforaphane and alpha-ketoglutaric acid can significantly prolong the days of PPS of the male mice; the addition of β-glucan alone (Plus1 regimen), β-glucan in combination with curcumin, vitamin K2, vitamin D3 and omega-3 fatty acids (Plus2 regimen), β-glucan in combination with curcumin, quercetin, vitamin K2, vitamin D3 and omega-3 fatty acids (Plus3 regimen), β-glucan in combination with curcumin, quercetin, melatonin, vitamin K2, vitamin D3 and omega-3 fatty acids (Plus4 regimen) and β-glucan in combination with curcumin, quercetin, melatonin, green tea polyphenols, vitamin K2, vitamin D3 and omega-3 fatty acids (Plus5 regimen) can all synergistically enhance this effect; and the Log-rank test was used for the comparison between groups in FIG. 18.

In FIGS. 15-18, ▴ indicates the comparison between the Control group and the HFD group; * indicates the comparisons between the HFD group and each of 6 groups, i.e., the HFD+ALA+SFN+AKG group, the HFD+ALA+SFN+AKG+Plus1 group, the HFD+ALA+SFN+AKG+Plus2 group, the HFD+ALA+SFN+AKG+Plus3 group, the HFD+ALA+SFN+AKG+Plus4 group and the HFD+ALA+SFN+AKG+Plus5 group; # indicates the comparisons between the HFD+ALA+SFN+AKG group and the HFD+ALA+SFN+AKG+Plus1 group, the HFD+ALA+SFN+AKG group and the HFD+ALA+SFN+AKG+Plus2 group, the HFD+ALA+SFN+AKG group and the HFD+ALA+SFN+AKG+Plus3 group, the HFD+ALA+SFN+AKG group and the HFD+ALA+SFN+AKG+Plus4 group, and the HFD+ALA+SFN+AKG group and the HFD+ALA+SFN+AKG+Plus5 group; ▴▴▴▴ or **** or #### refers to P<0.0001, ▴▴▴ or *** or ### refers to P<0.001, ▴▴ or ** or ## refers to P<0.01, and ▴ or * or # refers to P<0.05.

From the data shown in FIGS. 15-18, there is also a partial synergy between the combination of sulforaphane, alpha-lipoic acid and alpha-ketoglutaric acid in the active components of the present disclosure and the preferred third component (β-glucan in combination with curcumin, quercetin, melatonin, green tea polyphenols, vitamin K2, vitamin D3 and omega-3 fatty acids).

As can be seen from Example 1 and FIGS. 1-14, the active components (i.e., the first component and the second component) of the present disclosure can ameliorate the symptoms of precocious puberty in the female mice, and the combination of sulforaphane, alpha-lipoic acid and alpha-ketoglutaric acid has the best combined effect. According to Example 2 and FIGS. 15-18, the effect of ameliorating the symptoms of precocious puberty in the male mice is still achieved even when the amounts of the active components used are halved. Thus, it can be seen that the active components of the present disclosure can indeed ameliorate the symptoms of precocious puberty caused by obesity and estrogen secretion due to the high-fat diet (HFD). Example 2 explored that the third component was added to maintain the effect of the formula under the condition that the amounts of the first component and the second component used were halved. Although the third component is a common nutritional supplement or mineral, the combined addition of the third component can indeed enhance the amelioration of the symptoms of precocious puberty in the male mice and the symptoms of obesity and imbalance in hormone levels associated with this symptom, playing a role in optimizing the core active components.

The examples provided in the present disclosure are described in detail above. The principles and embodiments of the present disclosure are described herein with reference to specific examples, and the above description of the examples are merely intended to aid in understanding the core idea of the present disclosure. It should be pointed out that for those of ordinary skill in the art, several improvements and modifications can also be made to the present disclosure without departing from the principles of the present disclosure, and these improvements and modifications also fall within the protection scope of the claims of the present disclosure.

1. A composition for improving precocious puberty, comprising a first component and a second component, wherein
the first component comprises alpha-lipoic acid, and the second component comprises sulforaphane and/or alpha-ketoglutaric acid.

2. The composition for improving precocious puberty according to claim 1, wherein the first component comprises 100-300 parts by weight alpha-lipoic acid, and the second component comprises 10-400 parts by weight sulforaphane and/or 50-800 parts by weight alpha-ketoglutaric acid.

3. The composition for improving precocious puberty according to claim 1, wherein the second component comprises sulforaphane and alpha-ketoglutaric acid.

4. The composition for improving precocious puberty according to claim 1, further comprising a third component comprising β-glucan.

5. The composition for improving precocious puberty according to claim 2, further comprising a third component comprising β-glucan.

6. The composition for improving precocious puberty according to claim 3, further comprising a third component comprising β-glucan.

7. The composition for improving precocious puberty according to claim 4, wherein the composition comprises 500-4000 parts by weight β-glucan.

8. The composition for improving precocious puberty according to claim 5, wherein the composition comprises 500-4000 parts by weightβ-glucan.

9. The composition for improving precocious puberty according to claim 6, wherein the composition comprises 500-4000 parts by weightβ-glucan.

10. The composition for improving precocious puberty according to claim 4, wherein the third component further comprises one or more of melatonin, curcumin, quercetin, green tea polyphenols, vitamin and omega-3 fatty acids.

11. The composition for improving precocious puberty according to claim 5, wherein the third component further comprises one or more of melatonin, curcumin, quercetin, green tea polyphenols, vitamin and omega-3 fatty acids.

12. The composition for improving precocious puberty according to claim 6, wherein the third component further comprises one or more of melatonin, curcumin, quercetin, green tea polyphenols, vitamin and omega-3 fatty acids.

13. The composition for improving precocious puberty according to claim 10, wherein the vitamin comprises vitamin K2 and/or vitamin D3.

14. Use of a composition for improving precocious puberty, wherein the composition according to claim 1 is used for ameliorating symptoms of precocious puberty populations.

15. Use of a composition for improving precocious puberty, wherein the composition according to claim 2 is used for ameliorating symptoms of precocious puberty populations.

16. Use of a composition for improving precocious puberty, wherein the composition according to claim 3 is used for ameliorating symptoms of precocious puberty populations.

17. Use of a composition for improving precocious puberty, wherein the composition according to claim 1 is prepared into a food, a drug or a health product.

18. Use of acomposition for improving precocious puberty, wherein the composition according to claim 2 is prepared into a food, a drug or a health product.

19. Use of a composition for improving precocious puberty, wherein the composition according to claim 3 is prepared into a food, a drug or a health product.

20. The use according to claim 17, wherein the composition is prepared into a liquid beverage, a tablet, a capsule, a solid beverage or a functional food.
